(19) United States
(12) Patent Application Publication
(10) Pub. No.: US 2018/0140453 A1
(43) Pub. Date: May 24, 2018

(54) ORTHOSIS TO MITIGATE SCISSORING GAIT IN CEREBRAL PALSEY PATIENTS

(71) Applicants: THE JOHNS HOPKINS UNIVERSITY, Baltimore, MD (US); Kennedy Krieger Institute, Inc., Baltimore, MD (US)

(72) Inventors: Yu Xu, Baltimore, MD (US); Kevin Z. Xin, Baltimore, MD (US); Kaiyuan Wang, Baltimore, MD (US); Robert Allen, Baltimore, MD (US); Jacob Schick, Baltimore, MD (US); Tara Johnson, Baltimore, MD (US); Andie Seabrooke, Baltimore, MD (US); Pooja U. Nair, Baltimore, MD (US); Najwa Faqih, Baltimore, MD (US); Ana Ainechi, Baltimore, MD (US); Michael Ruiz, Baltimore, MD (US); Nathaniel J. Leon, Baltimore, MD (US)

(21) Appl. No.: 15/571,955

(22) PCT Filed: May 5, 2016
(86) PCT No.: PCT/US2016/030883

§ 371 Date: Nov. 6, 2017

Related U.S. Application Data
(60) Provisional application No. 62/157,036, filed on May 5, 2015.

(51) Int. Cl. A61F 5/01; A61F 5/37

(52) U.S. Cl. CPC A61F 5/0193; A61B 5/112; A61F 5/3715

(57) ABSTRACT

An embodiment in accordance with the present invention provides an orthotic device that provides a physical barrier between the thighs of the user. The orthotic device provides a physical barrier that separates the legs in order to minimize and prevent scissoring gait. The orthotic device includes specially designed blocks. The blocks separate the legs, fit comfortably against the legs, and slide back and forth against one another during the gait cycle to allow for walking movement. The blocks can be secured to the legs with straps. The straps can be exchangeable, so that a user can change them to match clothing or other color preferences. The orthotic device is useable in both indoor and outdoor settings.

### CROSS REFERENCE TO RELATED APPLICATIONS

This application claims the benefit of U.S. Provisional Patent Application No. 62/157,036 filed May 5, 2015, which is incorporated by reference herein, in its entirety.

### FIELD OF THE INVENTION

The present invention relates generally to medical devices. More particularly, the present invention relates to an orthosis to mitigate scissoring gait.

### BACKGROUND OF THE INVENTION

Cerebral palsy (CP) is a group of permanent, non-progressive movement disorders. Signs and symptoms vary, but often include poor coordination, stiff muscles, weak muscles, and tremors. In the United States, CP occurs in 2.1 out of every 1000 births. The classic symptoms are spasticity, spasms, other involuntary movements, unsteady gait, problems with balance. Scissoring gait (also called spastic diplegia) (where the knees come in and cross) and toe walking are common among people with CP. Currently, there are few method for treating scissoring gait and those that exist are cumbersome, uncomfortable, and relatively ineffective. For instance, treating scissoring gait can require lifelong physical therapy. Symptoms cannot be mitigated in many cases, even with the physical therapy. Scissor gait can also be caused by other neurological disorders, such as Multiple Sclerosis and spinal cord tumors.

Therefore, it would be advantageous to provide an orthosis to mitigate scissoring gait in cerebral palsy patients.

### SUMMARY OF THE INVENTION

The foregoing needs are met, to a great extent, by the present invention, wherein in one aspect an orthotic device includes a leg-spacer assembly comprising a right leg component and a left leg component. The right leg component is slideably coupled to the left leg component. A right-leg attachment structure is attached to the right leg component and structured to attach the right leg component of the leg-spacer assembly to a user's right leg. A left-leg attachment structure is attached to the left leg component and structured to attach the left leg component of the leg-spacer assembly to a user's left leg. The right and left leg components of the leg-spacer assembly are constrained to slide back-and-forth repeatedly with respect to each other during use.

In accordance with an aspect of the present invention, the right-leg attachment structure is a belt and buckle assembly, and the left-leg attachment structure is a belt and buckle assembly. The right leg component and the left leg component of the leg-spacer assembly each take the form of a low-friction material having a surface to contact each other. The right leg component and the left leg component of the leg-spacer assembly each include a foam portion to conform to a contact portion of the user's right and left legs. The right leg component and the left leg component of the leg-spacer assembly each have a plurality of modular components, such that a width of the leg-spacer assembly is adjustable. A connector assembly includes a rod extending between and slideably connecting the right leg component with the left leg component. The right leg component includes a channel in which the rod is slidably disposed. The left leg component includes a channel in which the rod is slidably disposed. The device can include first cover for the left leg component and a second cover for the right leg component. The device includes a dynamic sizing mechanism taking the form of a scissor jack device for expanding and contracting the size of the leg-spacer assembly. The dynamic sizing mechanism includes a turning key for engaging the expansion and contraction of the scissor jack device. A housing is configured to slidably expand and contract in conjunction with the expansion and contraction of the scissor jack device.

In accordance with another aspect of the present invention, the device includes suspension straps. The device can include markings on a surface of the leg-spacer assembly to indicate that the device is being worn correctly. The marking can take the form of halves of a shape, wherein one half is positioned on the left leg component and another half is positioned on the right leg component, and when the device is being worn correctly the halves of the shape are aligned. The device can include knee braces configured to be coupled to the left and right leg components. The knee braces can be secured with zippers. Expansion panels are added to the knee braces with zippers in order to customize the sizes of the knee braces. A casing is configured to be disposed over the left and right leg components for customization of the left and right leg components. The left-leg and right-leg attachment structures are exchangeable for different colors.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1A illustrates a perspective view of a device for mitigating scissoring gait, according to an embodiment of the present invention. FIG. 1B illustrates a partial view of the device for mitigating scissoring gait of FIG. 1A.

FIG. 2 illustrates a perspective view of a device for mitigating scissoring gait, and particularly the straps of the device, according to an embodiment of the present invention.

FIG. 3 illustrates a perspective view of an inner surface of a device for mitigating scissoring gait, according to an embodiment of the present invention.

FIGS. 5A and 5B illustrate partially exploded views of a device for mitigating scissoring gait, according to an embodiment of the present invention.

FIGS. 6A and 6B illustrate braces for use with a device for mitigating scissoring gait, according to an embodiment of the present invention.

FIGS. 7A and 7B illustrate a sectional view and a perspective view of leg components, according to an embodiment of the present invention.

FIGS. 8 and 9 illustrate variations in shape for the leg components, according to embodiments of the present invention.

FIG. 10 illustrates an embodiment of the device for mitigating scissoring gait, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides an orthotic device that provides a physical barrier between the thighs of the user. The orthotic device provides a physical barrier that separates the legs in order to minimize and prevent scissoring gait. The orthotic device includes specially designed blocks. The blocks separate the legs, fit comfortably against the legs, and slide back and forth against one another during the gait cycle to allow for walking movement. The blocks can be secured to the legs with straps. The straps can be exchangeable, so that a user can change them to match clothing or other color preferences. The orthotic device is useable in both indoor and outdoor settings.

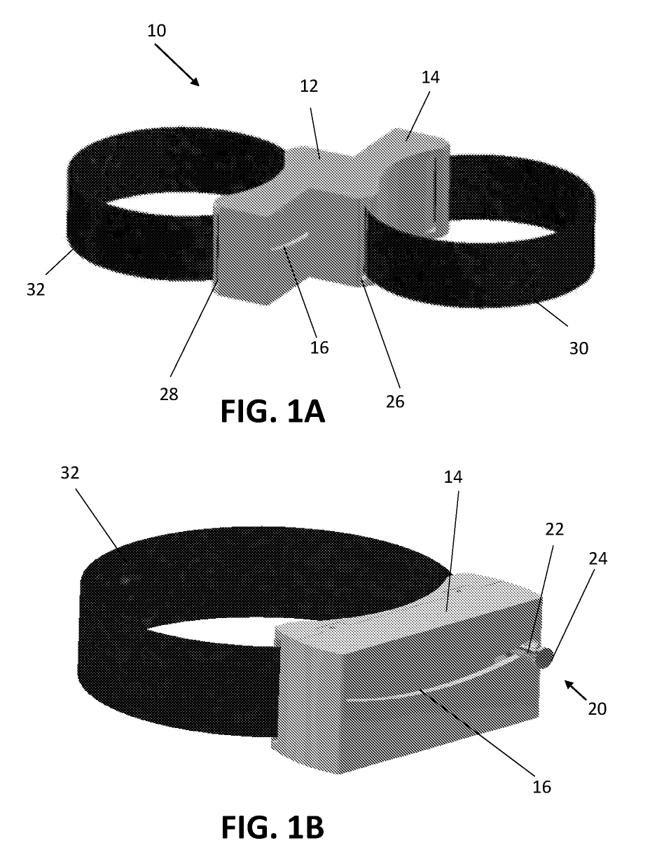

An orthotic device according to an embodiment of the current invention is illustrated in FIGS. 1A and 1B. The orthotic device in this embodiment includes a leg-spacer assembly that includes a right leg component and a left leg component. The right leg component is slideably connected to the left leg component. This embodiment also includes a right-leg attachment structure attached to the right leg component and structured to attach the right leg component of the leg-spacer assembly to a user's right leg; and a left-leg attachment structure attached to the left leg component and structured to attach the left leg component of the leg-spacer assembly to a user's left leg. The right and left leg components of the leg-spacer assembly are constrained to slide back-and-forth repeatedly with respect to each other during use. In the examples that follow, the right and left components can each be the one or more slabs with possible covering materials. The right-leg attachment structure and left-leg attachment structure can be, but are not limited to, belts with buckles and hook and loop strips as described below. Further, a rod structure can be used as a portion of an assembly for slideably connecting the right and left leg components.

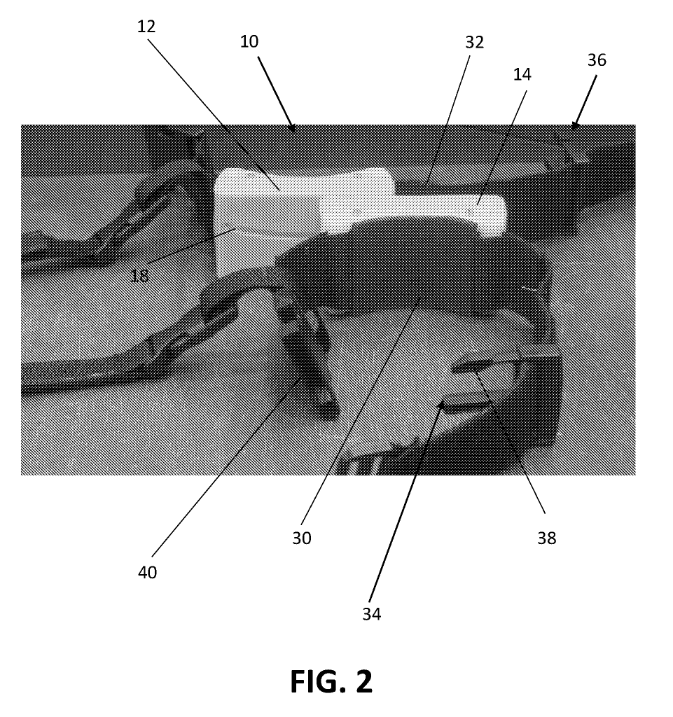

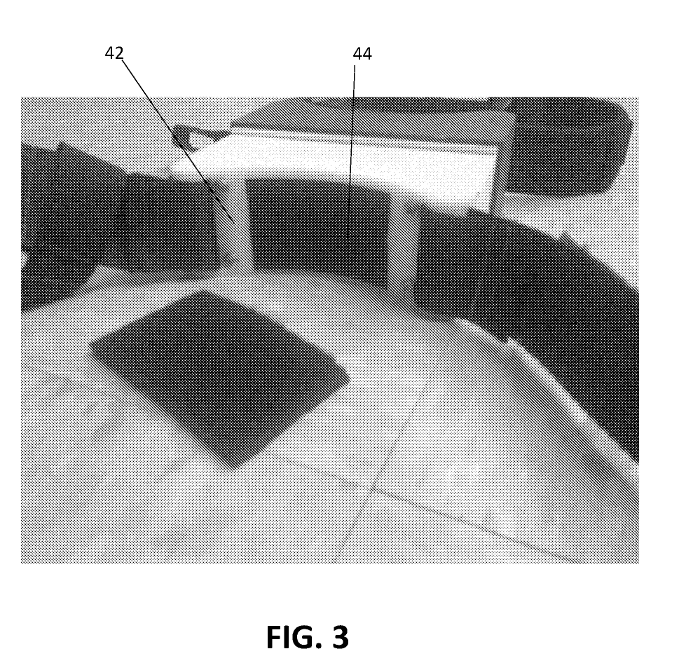

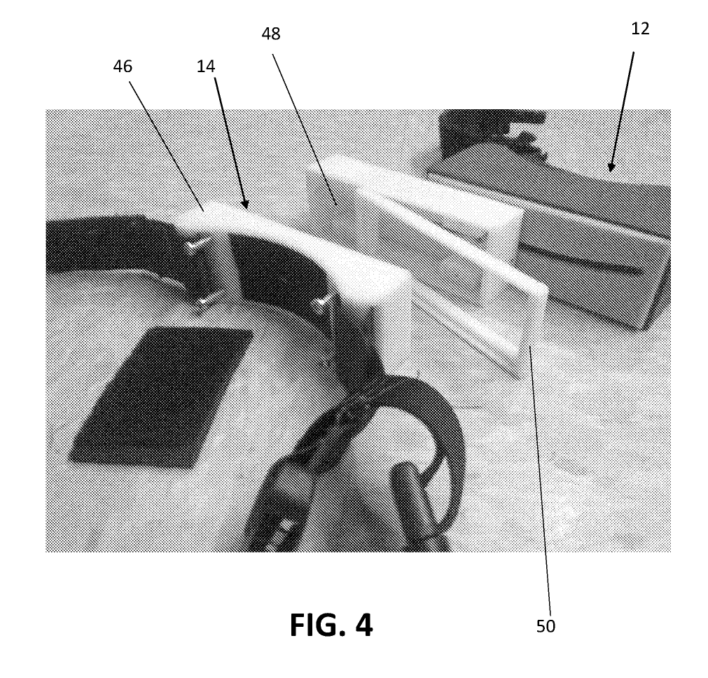

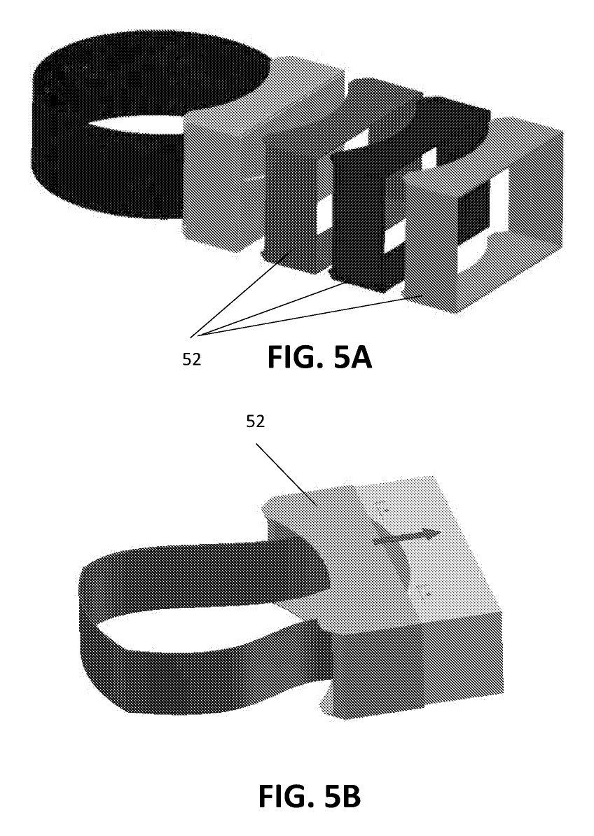

The following describes some embodiments in more detail. However, the general concepts of the current invention are not limited to the particular embodiments described.—The system of two slabs linked by a metallic rod has not been used previously in devices that are used to prevent scissoring during the gait cycle, as illustrated in FIGS. 1A and 1B. The straps can include buckles for quick release and hook and loop strips for adjustment, as illustrated in FIG. 2. The inner surface, which touches the thigh, can be covered with soft sponge rubber to improve patient comfort and the spongy pad can be detachable for cleaning, as illustrated in FIG. 3. Thickness of the blocks can be adjusted to accommodate patients of different body sizes, as illustrated in FIG. 4. The color of the outside casing can be changed to match the clothing by inserting and removing the casting from the rear of each half of the device, as illustrated in FIGS. 5A and 5B.

The orthosis according to some embodiments of the current invention utilizes a physical barrier (specially fabricated blocks) to separate the legs. The blocks in this embodiment are strapped onto each thigh, and are connected by a metallic bolt so the two blocks can easily slide along each other during the gait cycle, as illustrated in FIGS. 1A and 1B. The contact surface of the block can be covered with materials with a low friction coefficient, such as Teflon, to minimize friction. The inner surface, which touches the thigh, can be covered with soft sponge pads to improve patient comfort and the spongy pad can be detachable for cleaning, as illustrated in FIG. 3. The straps can be made of nylon and can include buckles for quick release and hook and loop strips for adjustment, as illustrated in FIG. 2. Thickness of the blocks can be adjusted to accommodate patients of different body sizes. This was achieved using frames (6 mm in thickness), which could be inserted into the device to increase its thickness. A maximum of three plastic frames can be added to each side of the device, hence enabling the thickness to increase by up to 36 mm, as illustrated in FIG. 4. However, the broad concepts of the current invention are not limited to this example. In addition, the color of the outside cover can be changed. This enables the device to match in color with the patient's clothing, thus making the device less conspicuous. The casing can be easily inserted and removed from the rear of each half of the device, as illustrated in FIGS. 5A and 5B.

FIG. 1A illustrates a perspective view of a device for mitigating scissoring gait, according to an embodiment of the present invention. FIG. 1B illustrates a partial view of the device for mitigating scissoring gait of FIG. 1A. As illustrated in FIG. 1A, the device for mitigating scissoring gait 10 includes a right leg component 12 and a left leg component 14. The right leg component 12 and the left leg component 14 are slidably coupled, such that the left leg component 12 and the right leg component 14 slide back and forth against one another. The right and left leg components 12 and 14 can include corresponding slots 16, 18. The slots 16, 18 (not pictured) are configured to hold a sliding bolt 20, illustrated in FIG. 1B. The sliding bolt 20 can include a middle portion 22, sized to slide along in slots 16, 18, and end portions 24 sized to hold the sliding bolt 20 within an interior space of the right and left leg components 12, 14. The sliding bolt 20 can be formed from a metal or other strong slideable material that can stand up to repeated sliding along the slots 16, 18 of the right and left leg components 12, 14. The right and left leg components 12, 14 can also include connectors 26, 28 for holding straps 30, 32. The straps 30, 32 hold the right and left leg components 12, 14 securely against the legs of the user. The straps can include buckles, hook and loop fasteners, or any other suitable method known to or conceivable by one of skill in the art for securing the straps 30, 32 and the right and left leg components 12, 14 in place. The right and left leg components 12, 14 can be formed from a metal, plastic, rubber, or other suitable material known to or conceivable by one of skill in the art. The material can be natural in finish or can include a paint or other coating in order to change the color, texture, or other aspects of appearance.

FIG. 2 illustrates a perspective view of a device for mitigating scissoring gait, and particularly the straps of the device, according to an embodiment of the present invention. As illustrated in FIG. 2, the device for mitigating scissoring gait 10 can include straps 30 and 32. In the embodiment illustrated in FIG. 2, the straps 30 and 32 include snap buckles 34, 36 to secure the device 10 on the user and to allow for quick release and removal. The snap buckles 34, 36 include a male component 38 and a female component 40, that are configured to couple together. The snap buckles 34, 36 can be configured to allow for the straps to be tightened and loosened, depending on the diameter of the leg of the user. The straps can be formed from an elastic material or have an adjustment device coupled to the strap for increasing and decreasing the size of the straps. The straps can be formed from a nylon or other suitable material. In some embodiments, the device is suspended by nylon straps attached to the elastic straps and attached to a patient's belt, or belt loops using a hook and loop fastening. The suspension system can be easily adjusted using plastic slides.

FIG. 3 illustrates a perspective view of an inner surface of a device for mitigating scissoring gait, according to an embodiment of the present invention. As illustrated in FIG. 3 an inner surface 42 of the right and left leg components is covered with a soft material 44. The soft material 44 improves patient comfort. The soft material 44 can be a spongy rubber, open-cell sponge rubber, plastic, fabric, or any other suitable material known to or conceivable by one of skill in the art. The soft material 44 can be permanently affixed to the right and left leg components, or it can be made removable and attached with a hook and loop fastener, or other suitable fastener, known to or conceivable by one of skill in the art. The padding spans the entire inner surface of the block for maximal comfort. For easy cleaning, the padding can be detached from the hook and loop fastener adhering it to the block. Additionally, there is a cover, preferably formed from cotton, which fits over the padding and can easily be removed and washed. The padding cover comes with an elastic band, so it can be stretched over the padding snugly.

FIG. 4 illustrates a partially exploded view of a device for mitigating scissoring gait, according to an embodiment of the present invention. As illustrated in FIG. 4, the right and left leg components 12, 14 can include a housing 46 that defines an interior space 48. The housing 46 can be formed from a number of pieces that fit together and couple frictionally, or by any other coupling known to or conceivable by one of skill in the art. Further with respect to FIG. 4, spacer blocks 50 can be incorporated into the housing 46. The spacer blocks 50 adjust the thickness of the right and left leg components 12, 14. The spacer blocks 50 can be easily adjusted in order to accommodate patients of different body sizes. The spacer blocks 50 can be added in any number and can be added to the right and/or the left leg components 12, 14. As illustrated in FIG. 4, the spacer blocks 50 come with a 6 mm diameter for incremental increase in width of the right and left leg components 12, 14. However, any suitable sizing for the spacer blocks 50 known to or conceivable by one of skill in the art can be used.

FIGS. 5A and 5B illustrate partially exploded views of a device for mitigating scissoring gait, according to an embodiment of the present invention. As illustrated in FIGS. 5A and 5B a color casing 52 can be added to the left and right leg components. The color casing 52 enables the device to match the color of the patient's clothing in order to be less conspicuous or to be a preferred color of the patient. The casing 52 can be easily applied and removed from the rear of each of the left and right leg components. Any other suitable way to apply and remove the casing 52 can also be used. The casing 52 can also include patterns, prints, designs or any other form of customization known to or conceivable by one of skill in the art.

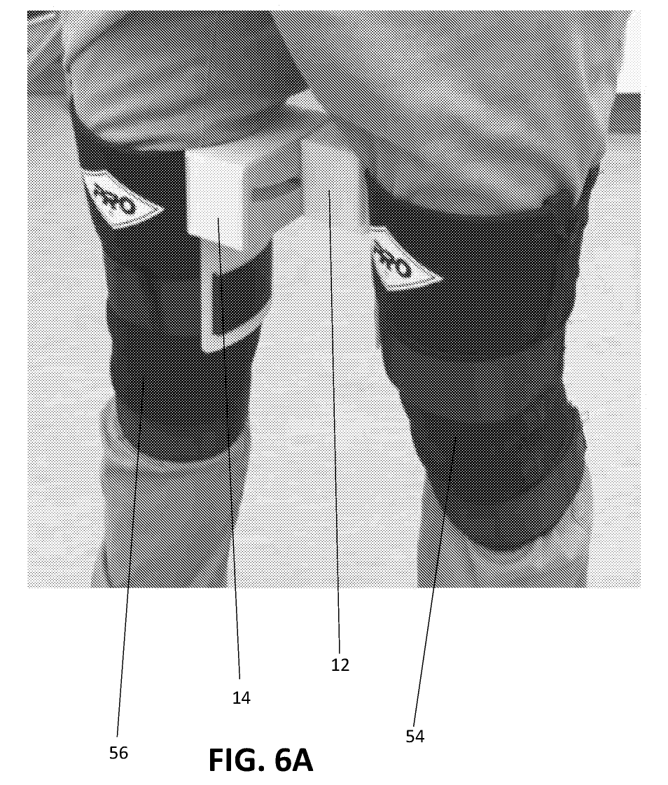

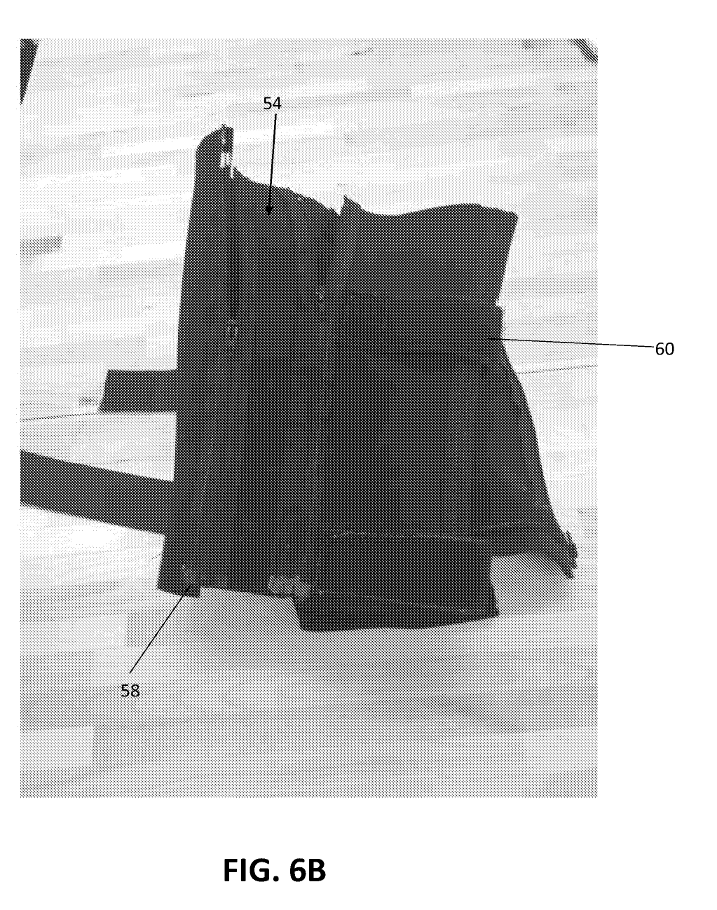

FIGS. 6A and 6B illustrate braces for use with a device for mitigating scissoring gait, according to an embodiment of the present invention. As illustrated in FIG. 6A braces 54, 56 are attached to the right and left leg components 12, 14. The braces 54, 56 take the form of a soft leg brace attached to the two blocks in place of the usual strap suspension system. The purpose of these braces 54, 56 is to use their tight fitting nature to prevent internal abduction of the thighs to improve mobility and joint health. The braces 54, 56 are constructed of a soft and elastic synthetic material such as nylon and polyester with a high degree of friction to prevent slipping of the braces laterally or radially on the thigh. As illustrated in FIG. 6B the brace 54 (and 56, not pictured) will feature a zipper 58 for quick and easy release and application of the brace 54 which can be supplemented by several hook and loop fastener straps 60 for added security and size adjustment. While the base size of the brace is fixed, strips of brace material flanked by additional zippers can be used to adjust the fit of the brace on the leg. The zippers on the insert will simply be zipped into the brace's existing zippers. The knee brace is secured with the adjustable straps above and below the knee joint. When first putting on the device, the legs need to be rotated outward at an appropriate angle (such that the feet point away from each other), and the outward orientation will be maintained by the straps.

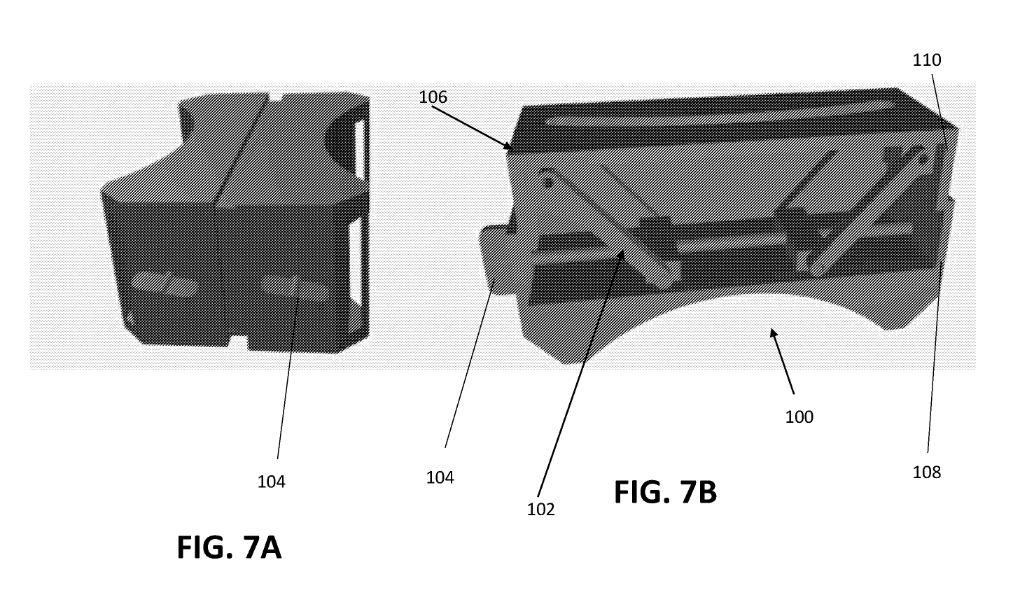

FIGS. 7A and 7B illustrate a sectional view and a perspective view of leg components, according to an embodiment of the present invention. As illustrated in FIG. 7A the leg component 100 can include a scissor jack device 102 configured to expand and contract the size of the leg component. As illustrate in FIGS. 7A and 7B, the scissor jack device 102 can include a key 104 that turns to expand and contract the scissor jack device 102 and in turn, the size of the leg component. The leg component 100 includes a housing 106 with a first component 108 and a second component 110. The first and second components 106 and 108 are slidably disposed against one another in order to expand and contract the size of the leg component 100. The scissor jack device 102 serves as a sizing device to help physicians determine an optimal width of the device for the patient's size and measurements. The scissor jack device 102 allows for easy adjustment for a quick and accurate fitting. The device with the scissor jack sizing component can be used in the clinical setting for sizing only and a permanently sized device can then be prepared for the patient. Alternately, the scissor jack device can include a lock for the key, so that it cannot be turned and the size of the device stays static for use by the patient.

Initial leg separation is determined by placing the sizable version of the device between the thighs, while the patient is standing with knee and hip joints aligned. A trial can then be performed using protocols such as GaitRight or the Six-Minute Walk test. Ability to walk and improvement of walking are measured by walking speed, swing phase percentage, and stride-time variability. Different sizes can be iteratively tested by the physician until the correct size is found. A general sizing guide can be included with the device in order to assist with general measurement ranges for patients of different sizes.

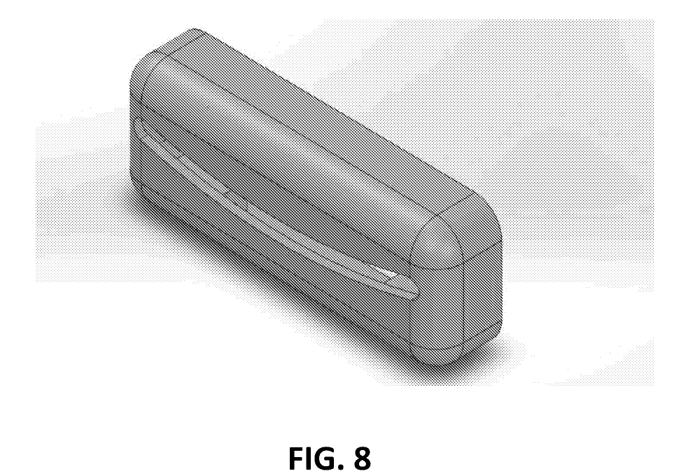

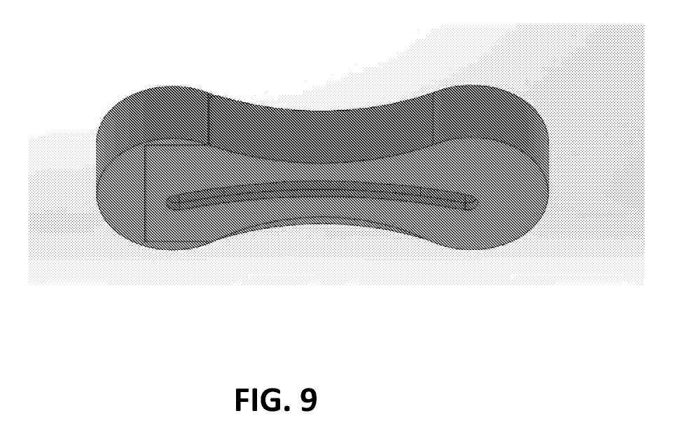

FIGS. 8 and 9 illustrate variations in shape for the leg components, according to embodiments of the present invention. Variations can be made in the shape of the design of the leg components without compromising function, in order to visually appeal to different users. The design of FIG. 8 is a sleek block shape. The design has rounded edges and is long and thin. The design is meant to be a sleek and discrete model of the device. The design of FIG. 8 is curvy and a bit chunkier than the design of FIG. 8 in order to allow for easier grip. The easier grip and the shape may appeal to younger children and those who need easier grip on the device.

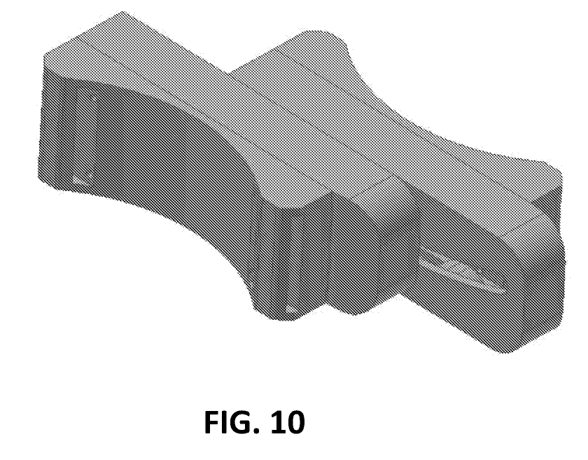

FIG. 10 illustrates an embodiment of the device for mitigating scissoring gait, according to an embodiment of the present invention. In some embodiments, it may be preferable to have a longer rail length with an elongated channel for the blocks to slide back and forth along. The device can also have varying options for rail and channel length for use by different sized patients and patients with different needs.

The left and right leg components can include marking to assist with the orientation of the device. In some embodiments the left and right leg components can include half of an equilateral triangle on the upper surface of the blocks. When the blocks are oriented correctly, the two triangle halves form a whole equilateral triangle. Any other suitable shape can be used.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. An orthotic device, comprising:
a leg-spacer assembly comprising a right leg component and a left leg component, wherein the right leg component is slideably coupled to the left leg component;
a right-leg attachment structure attached to the right leg component and structured to attach the right leg component of the leg-spacer assembly to a user's right leg; and
a left-leg attachment structure attached to the left leg component and structured to attach the left leg component of the leg-spacer assembly to a user's left leg, wherein the right and left leg components of the leg-spacer assembly are constrained to slide back-and-forth repeatedly with respect to each other during use.

2. The orthotic device of claim 1, wherein the right-leg attachment structure is a belt and buckle assembly, and wherein the left-leg attachment structure is a belt and buckle assembly.

3. The orthotic device of claim 1, wherein the right leg component and the left leg component of the leg-spacer assembly each comprise a low-friction material having a surface to contact each other.

4. The orthotic device of claim 1, wherein the right leg component and the left leg component of the leg-spacer assembly each comprise a foam portion to conform to a contact portion of the user's right and left legs.

5. The orthotic device of claim 1, wherein the right leg component and the left leg component of the leg-spacer assembly each have a plurality of modular components such that a width of the leg-spacer assembly is adjustable.

6. The orthotic device of claim 1, further comprising a connector assembly comprising a rod extending between and slideably connecting the right leg component with the left leg component.

7. The orthotic device of claim 6 wherein the right leg component includes a channel in which the rod is slidably disposed.

8. The orthotic device of claim 6 wherein the left leg component includes a channel in which the rod is slidably disposed.

9. The orthotic device of claim 1 further comprising a first cover for the left leg component and a second cover for the right leg component.

10. The orthotic device of claim 1 further comprising a dynamic sizing mechanism comprising a scissor jack device for expanding and contracting the size of the leg-spacer assembly.

11. The orthotic device of claim 10 further comprising a turning key for engaging the expansion and contraction of the scissor jack device.

12. The orthotic device of claim 10 further comprising a housing configured to slidably expand and contract in conjunction with the expansion and contraction of the scissor jack device.

13. The orthotic device of claim 1 further comprising suspension straps.

14. The orthotic device of claim 1 further comprising markings on a surface of the leg-spacer assembly to indicate that the device is being worn correctly.

15. The orthotic device of claim 14 further comprising the markings taking the form of halves of a shape, wherein one half is positioned on the left leg component and another half is positioned on the right leg component, and when the device is being worn correctly the halves of the shape are aligned.

16. The orthotic device of claim 1 further comprising knee braces configured to be coupled to the left and right leg components.

17. The orthotic device of claim 16 further comprising the knee braces being secured with zippers.

18. The orthotic device of claim 17 wherein expansion panels are added to the knee braces with zippers in order to customize the sizes of the knee braces.

19. The orthotic device of claim 1 further comprising a casing configured to be disposed over the left and right leg components for customization of the left and right leg components.

20. The orthotic device of claim 1 wherein the left-leg and right-leg attachment structures are exchangeable for different colors.
